(19) United States
(12) Patent Application Publication
(10) Pub. No.: US 2024/0191243 A1
(43) Pub. Date: Jun. 13, 2024

(54) HSV AMPLICON PACKAGING SYSTEM USING ENGINEERED CELLS

(71) Applicant: University of Pittsburgh - Of the Commonwealth System of Higher Education, Pittsburgh, PA (US)

(72) Inventors: Joseph C. Glorioso, III, Pittsburgh, PA (US); Justus B. COHEN, Allison Park, PA (US)

(21) Appl. No.: 18/286,847

(22) PCT Filed: Apr. 21, 2022
(86) PCT No.: PCT/US2022/025809

§ 371 Date: Oct. 13, 2023

Related U.S. Application Data
(60) Provisional application No. 63/177,682, filed on Apr. 21, 2021.

(51) Int. Cl. C12N 15/64; A61K 48/00; C12N 7/00; C12N 15/86

(52) U.S. Cl. CPC C12N 15/64; A61K 48/0091; C12N 7/00; C12N 15/86; C12N 2710/16622; C12N 2710/16643; C12N 2710/16652; C12N 2830/003

(57) ABSTRACT

Disclosed are custom HSV-1 packaging genomes for chromosomal integration into a cell line that is devoid of cis-acting HSV origins of replication (ori) and packaging sequences (pac) normally used for incorporation of the viral genome into virus particles, as well as the use of the resulting packaging cell for amplicon packaging.

### CROSS REFERENCE TO RELATED APPLICATION

This patent application claims the benefit of U.S. Provisional Patent Application No. 63/177,682, filed Apr. 21, 2021, the disclosure of which is incorporated by reference in its entirety herein.

### BACKGROUND OF THE INVENTION

Gene therapy has been rapidly developing as a treatment strategy for diseases affecting a variety of distinct metabolic, neurologic and cancer-related conditions. The challenge of gene therapy remains the creation of gene delivery vehicles that provide novel genetic information to different cell and tissue types and express the desired gene of interest at appropriate levels and duration. The gene therapy field has endeavored to take advantage of viral systems engineered to remove viral replication and cytotoxic functions. The bar for viral vectors is high since they must be safe and nonpathogenic, amenable to consistent manufacture, and capable of delivering a therapeutic payload to the correct tissue and cell type. The therapeutic gene must be expressed not only at the appropriate level, and thus be regulated either by transcriptional or post-transcriptional mechanisms, but also for an appropriate length of time in order to achieve maximal benefit. In addition, the therapy must persist without damage to the transduced cells or recruitment of immune responses that could eliminate corrected cells and shut down transgene expression.

There is a desire for new vector systems and packaging cell lines.

### BRIEF SUMMARY OF THE INVENTION

An aspect of the invention provides a packaging cell line comprising, consisting essentially of, or consisting of (a) a helper virus genome; (b) a nucleic acid sequence encoding reverse tetracycline transactivator (rtTA); (c) a nucleic acid sequence encoding VP16; (d) a nucleic acid sequence encoding ICP0; (e) a nucleic acid sequence encoding ICP4; and (f) a nucleic acid sequence encoding ICP27.

An aspect of the invention provides a method of producing herpes simplex virus (HSV) amplicon particles comprising transfecting the packaging cell line with an amplicon vector comprising, consisting essentially of, or consisting of an origin of replication (ori) and a packaging signal (pac), as well as at least one transgene.

An aspect of the invention provides an HSV amplicon particle produced by the method, as well as compositions, cells, and kits comprising the HSV amplicon particle.

An aspect of the invention provides a kit for preparing HSV amplicon particles comprising (i) the packaging cell line and (ii) an amplicon vector comprising an origin of replication (ori) and a packaging signal (pac).

An aspect of the invention provides a method of expressing a transgene in a cell comprising infecting the cell with the HSV amplicon particle, such that the at least transgene is expressed within the cell.

An aspect of the invention provides a method of treating a disease or condition in a subject, comprising administering an HSV amplicon particle to the subject in an amount and at a location sufficient to infect cells of the subject such that the at least one transgene is expressed in the subject.

### BRIEF DESCRIPTION OF THE SEVERAL VIEWS OF THE DRAWING(S)

FIG. 1 is a schematic showing amplicon particle generation. The left portion of FIG. 1 shows helper virus functions encoded by the packaging cell line and their induction by doxycycline (dox). The CAG promoter (CAGp) drives constitutive expression of rtTA which in the presence of dox activates the Tet-on promoter resulting in expression of the ICP0 and VP16 genes. VP16 in turn induces expression of the ICP4 and ICP27 genes from their cognate promoters (ICP4p, ICP27p) and together, VP16, ICP0, ICP4, and ICP27 initiate the transcription of the viral replication and structural genes from the integrated replication- and packaging-defective helper virus genome. Expression of these genes provides the components of the virion shell and enables rolling-circle replication of the transfected amplicon plasmid or minicircle DNA (right), producing linear arrays of packaging-competent payload with or without bacterial DNA. Bent arrows denote active transcription and lightning bolts denote cleavage at pac sites (triangles) during packaging.

### DETAILED DESCRIPTION OF THE INVENTION

Herpes Simplex Virus (HSV) is an enveloped virus containing a ˜152 kb double stranded DNA genome. HSV genes are expressed in waves, referred to as “cascade regulation”, producing first the immediate early (IE), then the early (E), and finally the late (L) gene products. The gene program is launched by a component of the infecting particle, the viral tegument protein VP16 delivered to the cell nucleus during infection that induces the expression of IE genes through binding to a viral enhancer sequence present in multiple copies within the IE gene promoters. Three critical IE genes are ICP0, ICP4, and ICP27. The IE gene products have multiple functions including transactivation of the early genes, mRNA transport, and interfering with innate host immune responses that include STING activation and IFN production. The early gene products are primarily responsible for viral genome replication whereupon the late genes are expressed that encode primarily structural proteins needed for DNA packaging and particle assembly. Many of the HSV genes are not essential for virus production per se but rather are important to carry out the complex life cycle in the host in vivo. HSV infects cells using an attachment/fusion entry mechanism minimally requiring 4 envelope glycoproteins (gB, gD, gH/gL) and virus production occurs in the host cell nucleus. Virus replication is lytic and newly formed particles acquire their envelope through nuclear membrane budding, Golgi processing, and envelope exchange at the cell surface. HSV in the latent phase can persist for life in sensory neurons of the peripheral nervous system, and in brain if infected. The viral genomes are maintained as episomes and their copy number can be quite high. During latency, viral lytic genes are largely silenced and only the noncoding latency associated transcript (LAT) gene is expressed. The virus is capable of reactivation from latency, which provides a means of virus spread to other individuals by direct contact with a recurrent lesion. Like HSV genomes in latency, replication-defective HSV and amplicon genomes remain extrachromosomal, but unlike latent HSV, in a cell-type independent manner. HSV vector technologies (including sequence information) are described in U.S. Pat. Nos. 5,658,724, 5,804,413, 5,849,571, 5,849,572, 5,879,934, 5,998,174, 6,261,552, 7,078,029, 7,531, 167, 10,174,341, and 10,201,575.

Current methods for HSV-mediated gene therapy are largely based on replication-defective vectors whereby essential HSV immediate early (IE) genes (plus other non-essential features) are replaced with therapeutic transgenes intended for long-term expression from the otherwise quiescent viral episomal genome. Replication-defective HSV vectors are similar to HSV amplicons in that both are designed to deliver and express transgene(s) in the absence of the normal HSV gene expression cascade, but differ in transgene payload capacity and viral gene content.

Specifically, while amplicons enable ˜150 kb of payload and carry no viral protein coding genes, replication-defective vectors provide at best ˜40 kb of transgene capacity given that the remainder of the genome encodes viral genes. It is not clear that all viral genes will remain silent under conditions that favor HSV reactivation and, thus, the complete elimination of all viral genes afforded in amplicon vectors improves the safety profile of the vector.

The genetic core of HSV amplicon vectors is composed of HSV replication and packaging signals embedded within up to 150 kb of designer payload DNA; smaller amplicon DNAs will be packaged as concatemers. Since this genetic core remains extrachromosomal, it poses no risk of insertional mutagenesis. Due to the simple design lacking any viral protein-coding genes, amplicon DNA packaging into viral particles completely depends on the presence of a helper virus, typically a replication-defective HSV-1, to minimize amplicon stock contamination with replication-competent HSV. In this instance, a complementing cell line is used that is engineered to express the essential replication genes that are absent from the helper virus genome.

The total reliance on helper viruses for amplicon production and packaging poses major manufacturing and validation challenges that have severely limited development of HSV amplicons for use in gene therapy. Specifically, high titer production leads to cross-contamination of amplicon stocks with variable amounts of defective helper virus particles, which are potentially cytotoxic and immunogenic. While HSV amplicons have been under development for years, to date a system for their production that excludes helper virus contamination has yet to be developed, and contamination levels have not been pushed below 0.1-1%. New technology development to meet safety and manufacturing requirements is desired to unlock the potential of high capacity HSV viral delivery for safe gene therapy with 0% contamination.

An aspect of the invention provides an effective packaging cell line for scalable production of safe, helper-free amplicon particles to solve current impediments to amplicon vector manufacture and clinical application, in particular where large transgene payloads are required. In particular, an aspect of the invention provides a custom HSV (e.g., HSV-1) packaging genome for chromosomal integration into a cell line (e.g., mammalian cell line). This packaging genome is devoid of cis-acting HSV origins of replication (ori) and packaging sequences (pac) normally used for incorporation of the viral genome into virus particles.

Specifically, an aspect of the invention provides a packaging cell line comprising, consisting essentially of, or consisting of (a) a helper virus genome; (b) a nucleic acid sequence encoding reverse tetracycline transactivator (rtTA); (c) a nucleic acid sequence encoding VP16; (d) a nucleic acid sequence encoding ICP0; (e) a nucleic acid sequence encoding ICP4; and (f) a nucleic acid sequence encoding ICP27.

In one aspect, the helper virus genome comprised within the packaging cell line does not contain an origin of replication (ori) or a packaging signal (pac). For example, the helper virus genome comprised within the packaging cell line does not contain oriL/S.

In some aspects, the helper virus genome comprised within the packaging cell line does not express one or more of ICP0, ICP4, and ICP27. In one aspect, the helper virus genome comprises an inactivating deletion of one or more (e.g., 1, 2, or 3) of the ICP0, ICP4, and ICP27 genes (e.g., inactivating mutations (deletions) of each of ICP0, ICP4, and ICP27). The inactivating deletion can be a deletion within, or of, the entire coding sequence of the ICP0, ICP4, and/or ICP27 gene or alternatively including the promoter or other regulatory sequences of such gene(s). In one aspect, the inactivating deletion can be a complete deletion of the coding sequence of one or more of ICP0, ICP4, and ICP27, such that the helper virus genome does not contain nucleic acid sequences of the ICP0, ICP4, and ICP27 genes.

In some aspects, the helper virus genome does not contain an internal repeat region (joint), such as a joint comprising IRs and IRL. By excluding the joint, one copy each of the immediate early genes ICP0 and ICP4 are eliminated, as well as the promoter for the ICP22 or ICP47 immediate early gene.

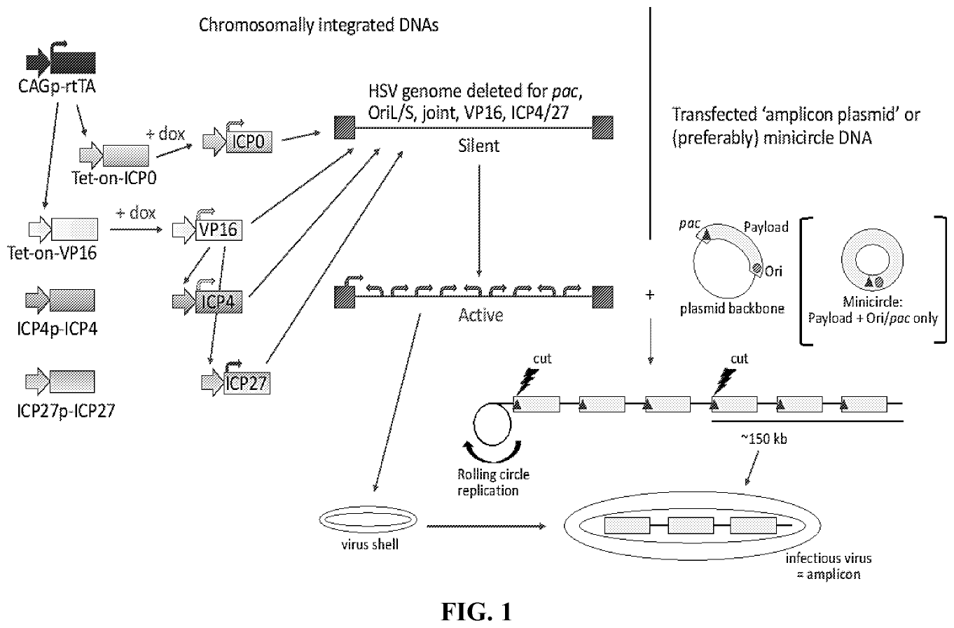

In one aspect, the helper virus genome comprised within the packaging cell line does not contain sequences for VP16, ICP4, ICP27, or any combination thereof. In particular, the helper virus genome does not contain ori/pac sequences (oriL/S); sequences encoding VP16, ICP0, ICP4, and ICP27; or the joint (see FIG. 1).

The helper virus genome can encode one or more (two, three, four, five, or any range of these values) glycoprotein(s) that target the amplicon for selective infection of particular cell types. For example, the helper genome can encode a viral glycoprotein that is defective for recognition of the natural viral cognate receptors (i.e., fully detargeted). The glycoprotein is modified to contain a ligand placed in frame within the viral glycoprotein that enables virus attachment and entry into a selected cell type.

The packaging cell line comprises a nucleic acid sequence encoding reverse tetracycline transactivator (rtTA). In particular, the packaging cell line can comprise a gene cassette for expression (e.g., constitutive expression) of rtTA. rtTA combines a variant of the DNA binding domain from the TetR regulator of the bacterial tetracycline resistance transposon with a highly efficient viral transcriptional activation domain (Roney et al., Scientific Reports, 6:27697 DOI: 10.1038/srep27697 (2016)). Upon the addition of tetracycline, rtTA undergoes a conformational change that increases its affinity for a unique 19 base-pair DNA binding site and enhances the recruitment of the machinery required for transcription (Roney et al., Scientific Reports, 6:27697 DOI: 10.1038/srep27697 (2016)). The rtTA system offers many advantages because it enables graded and reversible transcriptional control using a non-toxic inducer, the antibiotic doxycycline, that has few pleiotropic effects (Roney et al., Scientific Reports, 6:27697 DOI: 10.1038/srep27697 (2016)). The Tet-On system is described in, e.g., Das et al., Curr. Gene Ther., 16(3): 156-167 (2016)).

In one aspect, rtTA is under the control of a eukaryotic promoter, such as a constitutive mammalian promoter (e.g., an SV40, RSV, CMV, ubiquitin C (UbC), CAG, or β-actin promoter, etc.). rtTA preferably is under the control of the CAG (CMV enhancer/chicken beta-actin promoter/chimeric intron) promoter.

The packaging cell line comprises a nucleic acid sequence encoding VP16. The nucleic acid sequence encoding VP16 can be under the control of a Tet-On system (rtTA+dox) in the packaging cell line. In one aspect, VP16 is expressed as a fusion polypeptide comprising a reporter/marker to monitor induction. For example, VP16 can be expressed as fusion polypeptide comprising green fluorescent protein (GFP) (e.g., a VP16-2A-GFP fusion).

The packaging cell line comprises a nucleic acid sequence encoding ICP0. The nucleic acid sequence encoding ICP0 can be under the control of a Tet-On system.

The packaging cell line also comprises nucleic acid sequences encoding ICP4 and ICP27. In one aspect, one or both of these sequences can be under the control of the respective cognate viral promoters (i.e., the natural VP16 responsive promoters) in the packaging cell line. As such, these genes may remain silent until the HSV tegument protein VP16 appears in the nucleus, where it promotes high-level expression of the integrated ICP4 and ICP27 genes by activation of their promoters.

Each of the recited components of the packaging cell line (i.e., (a) a helper virus genome; (b) a nucleic acid sequence encoding rtTA; (c) a nucleic acid sequence encoding VP16; (d) a nucleic acid sequence encoding ICP0; (e) a nucleic acid sequence encoding ICP4; and (f) a nucleic acid sequence encoding ICP27) are integrated into the genome of the packaging cell line at the same or different locations. Safe harbor sites in the mammalian genome are described in, for example, Mitchell et al., J. Vis. Exp., 99: e52941 (2015); and Papapetrou et al., Mol. Ther., 24(4): 678-684 (2016).

For example, the ICP0 nucleic acid sequence under the control of the Tet-On promoter can be introduced into a safe harbor site of the packaging cell genome for simultaneous induction of VP16 and ICP0 production. ICP0 expression is essential for maximum helper virus gene expression but must be off during growth of the packaging cell line since the protein is toxic. Additionally or alternatively, the ICP4 nucleic acid sequence, ICP27 nucleic acid sequence, helper virus genome, and/or VP16 nucleic acid sequence are introduced into a safe harbor site of the packaging cell genome. The safe harbor sites can be the same or different.

The packaging cell line can be any suitable cell line (e.g., a mammalian cell line).

The packaging cell line can be engineered to express a gene encoding a selectable marker, such as markers typically employed in engineering packaging cells or cells expressing any other foreign gene. Suitable selectable genes include those conferring resistance to neomycin/G418, hygromycin, blasticidin, puromycin, zeocin, and the like. Additionally or alternatively, the packaging cell line can be engineered to express LacZ (encoding beta-galactosidase), CAT (encoding chloramphenicol acetyltransferase), or a fluorescent protein-encoding gene (e.g., GFP, YFP, RFP, and analogues thereof such as iRFP, EGFP, and the like).

Any suitable method for engineering a source cell type to contain the recited components of the packaging cell line (i.e., (a) a helper virus genome; (b) a nucleic acid sequence encoding rtTA; (c) a nucleic acid sequence encoding VP16; (d) a nucleic acid sequence encoding ICP0; (e) a nucleic acid sequence encoding ICP4; and (f) a nucleic acid sequence encoding ICP27) as well as other gene products (such as the selectable gene) can be used.

The packaging cell line can be propagated and cloned. Thus, an aspect of the invention provides a clonal population, i.e., a cell line, comprising or consisting of or essentially of the packaging cell line.

Using the packaging cell line, an HSV amplicon particle can be propagated. Therefore, an aspect of the invention provides a method of producing HSV (HSV-1) amplicon particles comprising transfecting the packaging cell line with an amplicon vector comprising an origin of replication (ori) and a packaging signal (pac). The number of HSV amplicon particles can be amplified by passaging. The method can further comprise isolating the HSV amplicon particles from the packaging cell line.

As shown in FIG. 1 (right), the amplicon vector can be an amplicon plasmid comprising ori/pac sequences and a plasmid backbone, or minicircle DNA comprising ori/pac sequences. Each of the amplicon plasmid and minicircle additionally comprises at least one transgene (i.e., payload as shown in FIG. 1). Therefore, the amplicon vector comprises, consists essentially of, or consists of ori/pac sequences and at least one transgene.

The ori/pac sequences (i.e., HSV ori/pac sequences) for use in the amplicon vector can be any suitable sequences. The ori/pac sequences can be placed into the genome of the amplicon vector in any suitable locations.

In particular, the HSV origin of replication can be any suitable origin of replication that allows for replication of the amplicon vector in the packaging cell line used for replication and packaging of the vector into the HSV amplicon particles. For example, origin of replication signals from HSV-1 (or HSV-2) can be used. In another aspect, the origin of replication can be a mammalian ori, which leads to amplicon persistence in dividing cells. Any suitable mammalian ori can be used, including those described in Hamlin, Bioessays, 14(10): 651-9 (1992).

The pac sequence for use in the amplicon vector can be any suitable pac sequence, such that the amplicon vector can be packaged into a HSV amplicon particle that is capable of adsorbing to a cell (i.e., which is to be transformed or transduced). Exemplary pac sequence include pac sequences from HSV-1 and HSV-2.

The transgene(s) can contain a promoter sequence and a transcribed sequence such that the transcribed sequence(s) is controlled by the promoter. The promoter within the transgene can be any promoter desired to control/regulate the expression of the transcribed sequence(s). For example, the promoter can be a cell-specific or tissue-specific promoter (e.g., EOS, OCT4, Nanog (for ESC/iPSC), SOX2 (for neural stem cells), αMHC, Brachyury, Tau, GFAP, NSE, Synapsin I (for neurons), Apo A-I, Albumin, ApoE (for liver), MCK, SMC α-Actin, Myosin heavy chain, Myosin light chain (for muscle), etc.), such as a promoter that specifically or preferentially expresses genes in a defined cell type (e.g., within a liver cell, lung cell, epithelial cell, cardiac cell, neural cell, skeletal muscle cell, embryonic, induced pluripotent, or other stem cell, cancer cell, etc.). Promoters for use in sensory neurons include TRPV1, CGRP, and NF200. The promoter within a transgene expression cassette can be a constitutive mammalian promoter (e.g., SV40, CMV, CAG, EF1α, UbC, RSV, β-actin, PGK, and the like). Depending on the activity of the promoter within the transgene, the transgene can be expressed in any type of mammalian (especially human) cell that can be infected without the cytotoxicity associated with viral gene expression.

In other aspects, the promoter can be an inducible promoter, such as a Tet-On promoter (e.g., TRE3G). When a Tet-On promoter is used, expression of its cognate rtTA is needed. For example, use of TRE3G as the promoter necessitates expression of the cognate dox-sensitive transactivator protein, rtTA3G. The cognate rtTA can be expressed from the amplicon or from target cells or tissue (e.g., by introduction of a viral (e.g., AAV) vector encoding the rtTA into the target cells or tissue).

The transgene(s) also can comprise additional regulatory element(s). For example, the transgene(s) can include one or more sites for binding of microRNA. In one aspect, the transgene(s) comprise tandem binding sites for such microRNAs, such as 2, 3, 4, 5, or 6 tandem sites (four being typical). The presence of such sites, particularly tandem binding sites for such microRNAs, facilitates down-regulation of the transgene expression in certain cell types. Thus, for example, a transgene for expression in a cancer or tumor cell (which may be toxic to many cell types) can comprise binding sites for microRNAs of “normal” (i.e., non-malignant) cells, so that the expression of the transgene is suppressed in non-malignant cells.

The transgene(s) can be monocistronic (i.e., encoding a single protein or polypeptide) or polycistronic (i.e., encoding multiple proteins or polypeptides). All or part of the transcribed portion of the transgene(s) also can encode non-translated RNA, such as siRNA or miRNA. Multiple separate monocistronic or polycistronic transgene units (preferably two separate transgene units but possibly more (e.g., three, four, five, or more separate units)), each with its own respective promoter, translated sequence(s) or non-translated RNA sequence(s), and other regulatory elements can be used. In one aspect, the transgene is a genomic gene that includes the gene's respective promoter, as well as introns and other regulatory sequences.

The transgene can comprise a gene editing system such CRISPR/Cas9 or nucleases, such as meganuclease, TALENS, zinc finger nuclease (ZFN), and Cpf1 DNA endonuclease. In one aspect, the gene editing system can be used for the modification of epigenetic structures to silence genes or induce the expression of silent genes.

The transgene(s) include one or more (e.g., one, two, three, four, five, six, seven eight nine, ten, or ranges of any of these values) transcribed sequence(s), which are expressed under the control of the promoter and optionally other regulatory elements within the transgene (e.g., including an operable connection to insulator sequence(s)).

Any suitable insulator sequence(s) can be used, including AT-rich insulator-like sequences described in Soukupova et al., Molecular Therapy: Methods & Clinical Development, 21: 399-412 (2021), the Drosophila Gypsy insulator described in Ebersole et al., Cell Cycle, 10(16): 2779-2791 (2011), the chicken-β-globin insulator (e.g., chicken β-globin HS4 fragment (cHS4)) described in Ebersole et al. and de Silva et al., Viruses, 1(3): 594-629 (2009), and tRNA described in Ebersole et al. The AT-rich insulator-like sequences described in Soukupova et al. were shown to protect the transgene from silencing (i.e., from losing transgene expression) following delivery to the brain. Ebersole et al. demonstrated that copies of mouse tRNA genes are effective barrier elements, wherein the number of tRNA genes as well as their orientation can influence barrier function. Replacement of intervening and flanking regions of tRNA genes with AT-rich sequences resulted in extended maintenance of barrier activity. While not wishing to be bound by any particular theory, it is believed that the use of AT-rich sequences in insulator elements minimizes promoter methylation, thereby preventing silencing of transgene expression.

A transcribed sequence can be any sequence desired to be expressed within a given cell. Non-limiting examples of transcribed sequences that can be present in a transgene within the inventive vector include Oct4, Klf4, Sox2, c-Myc, L-myc, dominant-negative p53, Nanog, Glis1, Lin28, TFIID, GATA4, Nkx2.5, Tbx5, Mef2C, Myocd, Hand2, SRF, Mesp1, SMARCD3, SERCA2a, Pax3, MyoD, Lhx2, FoxG1, FoxP2, Is11, Ctip2, Tbr1, Ebf1, Gsx2, Srebp2, Factor VIII, Factor IX, Dystrophin, CFTR, GlyRα1, enkephalin, GAD67 (or other GAD isoforms, e.g., GAD 65), TNFα, interleukins (e.g., IL-4 or IL-12), Cas9 or other nucleases as described herein, a neurotrophic factor (e.g., NGF, BDNF, GDNF, NT-3), Ascl1, Nurr1, Lmx1A, Brn2, Myt11, NeuroD1, FoxA2, Hnf4α, Foxa1, Foxa2 or Foxa3, anti-checkpoint antibodies, any microRNA or combination of miRNAs (e.g., hsa-mir-302/367 gene cluster; hsa-miR200c; hsa-miR369; hsa-mir-124) and/or one or more other non-coding RNAs (“ncRNA(s)”) or a reporter gene for expression in mammalian cells, such as LacZ (encoding beta-galactosidase), CAT (encoding chloramphenicol acetyltransferase), luciferase, or a fluorescent protein-encoding gene (e.g., GFP, YFP, RFP, and analogues thereof such as iRFP, EGFP, and the like).

The plasmid backbone in the amplicon vector can comprise a bacterial artificial chromosome (BAC) cassette. The inclusion of such BAC cassette facilitates propagation and manipulation of the amplicon vector within bacteria. The BAC cassette can include bacterially-expressed sequences that assist in the use of bacterial strains, e.g., selectable genes, such as genes conferring bacterial resistance to antibiotics or toxins (e.g., preferably chloramphenicol, but other resistance genes (e.g., for tetracycline, ampicillin, zeocin, etc.) can also be employed). The BAC cassette can further include reporter genes (e.g., LacZ (encoding beta-galactosidase), or a fluorescent protein-encoding gene (e.g., GFP encoding green fluorescent protein, YFP encoding yellow fluorescent protein, RFP encoding red fluorescent protein, and analogues thereof (e.g., encoding iRFP, EGFP, and the like)) under the control of a eukaryotic promoter, such as a constitutive mammalian promoter (e.g., an SV40, RSV, CMV, ubiquitin C (UbC), CAG, or β-actin promoter, etc.).

The BAC cassette can be placed into the genome of the amplicon vector in any suitable location. The BAC cassette can be flanked by sequences facilitating removal of the BAC cassette, such as by site-specific recombinase recognition sites/consensus sequences (e.g., those recognized by enzymes such as cre, dre, flp, KD, B2, B3, R, etc.). The inclusion of such sites facilitates excision of the BAC cassette, if desired, since BAC sequences have been shown to reduce virus growth in cultured cells, stimulate an innate response, and promote transgene silencing in infected cells. Excision of the BAC cassette also can increase the capacity for the vector to incorporate one or more transgenes, since BAC cassettes are on the order of about 11 kb. The amplicon vector can have one or more (e.g., one, two, three, four, or five) consensus recognition sequences for a recombinase enzyme (e.g., loxP), particularly one not native to the HSV genome. Removal of a BAC cassette using a cell line that expresses an appropriate site-specific recombinase for excising the BAC cassette leaves a single copy of the one or more consensus sequences for a recombinase enzyme within the amplicon genome. As described in Example 2, the bacterial sequences also can be eliminated by inducible integrase-mediated “looping-out” and selective degradation of the bacterial elements flanked by compatible att sites, similar to plasmid-derived minicircle production but requiring a BAC-compatible minicircle E. coli strain.

The HSV amplicon can be present as isolated DNA, DNA within a cell, or packaged in a viral envelope (HSV amplicon particle). Upon transfection of the amplicon DNA into the packaging cell line, infectious amplicon particles comprising, consisting essentially of, or consisting of one or more (e.g., one, two, three, four, or ranges of any of these values) tandem copies of the amplicon DNA surrounded by the HSV capsid, tegument, and envelope proteins are produced (see Example 1).

An aspect of the invention also provides an HSV amplicon particle produced by the inventive production method, as well as compositions and cells comprising the HSV amplicon particle.

An aspect of the invention provides a method of expressing a transgene in a cell (e.g., a nucleated cell) comprising infecting the cell with an HSV amplicon (e.g., HSV amplicon particle), such that the at least transgene is expressed within the cell. In accordance with the method, the HSV amplicon is exposed to the cell under conditions suitable for the HSV amplicon to infect the cell. Once the cell is infected, the transgene will be transcribed (expressed) within the cell, provided the promoter within the transgene is one which is active in the cell and that the transgene is not suppressed by another regulatory mechanism. In other words, the HSV amplicons serve as gene transfer and expression vectors within cells (e.g., mammalian cells).

An aspect of the inventive method can be employed to express one or more (e.g., one, two, three, four, five, six, seven, eight, nine, ten, or ranges of any values thereof) transgene(s) within cells either in vivo or in vitro, as desired. For use in vivo, the cell (i.e., the cell comprising the HSV amplicon) can be any type of desired cell, such as exocrine secretory cells (e.g., glandular cells, such as salivary gland cells, mammary gland cells, sweat gland cells, digestive gland cells, etc.), hormone secreting gland cells (e.g., pituitary cells, thyroid cells, parathyroid cells, adrenal cells, etc.), ectoderm-derived cells (e.g., keratinizing epithelial cells (e.g., making up the skin and hair), wet stratified barrier epithelial cells (e.g., of the cornea, tongue, oral cavity, gastrointestinal tract, urethra, vagina, etc.), cells of the nervous system (e.g., peripheral and central neurons, glia, etc.)), mesoderm-derived cells, cells of many internal organs (such as kidney, liver, pancreas, heart, lung) bone marrow cells, and cancerous cells either within tumors or otherwise. Non-limiting examples of cells suitable for use in an aspect of the invention include liver cells, lung cells, epithelial cells, cardiac cells, muscle cells, stem cells, and cancer cells.

When used in vivo, the inventive method can treat a disease or a condition within a subject, when the transgene within the vector encodes one or more prophylactically- or therapeutically-active proteins, polypeptides, or other factors (e.g., non-coding RNA (ncRNA) such as siRNA or miRNA). Thus, an aspect of the invention provides a method of treating a disease or condition in a subject comprising administering the HSV amplicon to the subject in an amount and at a location sufficient to infect cells of the subject, such that the transgene is expressed within the cells of the subject, and wherein the transgene encodes one or more prophylactically or therapeutically active proteins, polypeptides or ncRNA. For example, the disease or condition can be a type of cancer, in which the transgene can encode an agent that enhances tumor killing activity (such as TRAIL or tumor necrosis factor (TNF)). As additional non-limiting example, the transgene can encode an agent suitable for the treatment of conditions such as muscular dystrophy (a suitable transgene encodes Dystrophin), cardiovascular disease (suitable transgenes include, e.g., SERCA2a, GATA4, Tbx5, Mef2C, Hand2, Myocd, etc.), neurodegenerative disease (suitable transgenes include, e.g., NGF, BDNF, GDNF, NT-3, Huntingtin, etc.), chronic pain (suitable transgenes encode GlyRα1, an enkephalin, or a glutamate decarboxylase (e.g., GAD65, GAD67, or another isoform), lung disease (e.g., CFTR), or hemophilia (suitable transgenes encode, e.g., Factor VIII or Factor IX).

In other aspects, the inventive method can be used in vitro to cause expression of the transgene within cells in culture. Any type of cells can be infected in vitro, such as stem cells and fibroblasts (e.g., human dermal fibroblasts (HDF) or human lung fibroblasts (HLF)). Other non-limiting types of cells for use in vitro include keratinocytes, peripheral blood mononuclear cells, hematopoietic stem cells (CD34+), or mesenchymal stem/progenitor cells. In one aspect, the transgene(s) encode one or more factors related to the differentiation of the cell. For example, expression of one or more of Oct4, Klf4, Sox2, c-Myc, L-Myc, dominant-negative p53, Nanog, Glis1, Lin28, TFIID, mir-302/367, or other miRNAs can cause the cell to become an induced pluripotent stem (iPS) cell. Alternatively, the transgene(s) can encode a factor for transdifferentiating the cells (e.g., one or more of GATA4, Tbx5, Mef2C, Myocd, Hand2, SRF, Mesp1, SMARCD3 (for cardiomyocytes); Ascl1, Nurr1, Lmx1A, Brn2, Myt11, NeuroD1, FoxA2 (for neural cells), Hnf4α, Foxa1, Foxa2, or Foxa3 (for hepatic cells).

The inventive methods can be used to express exogenous genes or supplement for deficient genes in animals (e.g., mammals) such as mice, rats, guinea pigs, hamsters, cats, dogs, cattle, horses, sheep, goats, swine, and the like. The inventive method can be used in vivo in humans as well, to provide for the expression of a prophylactically- or therapeutically-active agent, or factor. The factor (supplied by expression of one or more of the transgenes) can be exogenous, or one that complements a genetic deficiency.

The HSV amplicon particle can be administered alone or in a composition (e.g., pharmaceutical composition) that can comprise at least one carrier (e.g., a pharmaceutically acceptable carrier). The HSV amplicon particle or composition can be administered by any suitable route, including parenteral (e.g., subcutaneous, intravenous, intraarterial, intramuscular, intradermal, interperitoneal, and intrathecal), topical, oral, aerosol, rectal, vaginal, and local administration.

The pharmaceutically acceptable carrier (or excipient) is preferably one that is chemically inert to the HSV amplicon particle and one that has no detrimental side effects or toxicity under the conditions of use. Such pharmaceutically acceptable carriers include, but are not limited to, water, saline, Cremophor EL (Sigma Chemical Co., St. Louis, MO), propylene glycol, polyethylene glycol, alcohol, and combinations thereof. The choice of carrier will be determined in part by the HSV amplicon particle, as well as by the particular method used to administer the composition. Accordingly, there is a wide variety of suitable formulations of the composition.

The exact amount of the HSV amplicon particles or compositions thereof will vary from subject to subject, depending on the species, age, weight and general condition of the subject, the severity of the disease being treated, the particular virus or vector used, its mode of administration and the like. Thus, it is not possible to specify an exact amount for every composition. However, an appropriate amount can be determined by one of ordinary skill in the art using only routine experimentation given the teachings herein. Typically, a composition will contain at least about 1×10⁷ amplicon particles/ml (e.g., 1×10⁷, 1×10⁸, 1×10⁹, 1×10¹⁰ amplicon particles/ml, 1×10¹¹ amplicon particles/ml, and 1×10¹² amplicon particles/ml as well as any ranges of values thereof).

Also provided are kits comprising the HSV amplicon particles described herein and kits for preparing HSV amplicon particles. A kit for preparing HSV amplicon particles comprises an amplicon vector comprising an HSV origin of replication and an HSV packaging signal. A kit can further include instructions for use, a container, an administrative means (e.g., a syringe), other biologic components such as one or more cells and the like. The amplicon vectors can comprise one or more of the components described herein (e.g., one or more transgenes).

While not wishing to be bound by any particular theory, in the inventive HSV amplicon packaging system, chemical induction activates the integrated silent packaging (helper) genome to express viral DNA polymerase and other genes needed for replication of transfected, ori/pac-containing amplicon DNA. The helper virus genome in the packaging cell line is activated by expression of VP16 (which can be independently integrated at a separate location in the packaging cell genome). VP16 is responsible for activation of the immediate early genes of the virus that launch the cascade of gene expression of the rest of the viral genes within the integrated helper virus genome. In one aspect, VP16 expression is under control of a Tet-On system and, thus, can be induced by treatment of the cells with doxycycline (see FIG. 1).

The system can be pre-activated prior to amplicon DNA transfection and in readiness for amplicon particle production. Newly formed amplicon particles can subsequently be amplified by infection of the packaging cell line without drugs since both VP16 and ICP0 will be provided by the amplicon as components of its shell.

Aspects, including embodiments, of the subject matter described herein may be beneficial alone or in combination, with one or more other aspects or embodiments. Without limiting the foregoing description, certain non-limiting aspects of the disclosure numbered 1-38 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
(1) A packaging cell line comprising:
   (a) a helper virus genome;
   (b) a nucleic acid sequence encoding reverse tetracycline transactivator (rtTA);
   (c) a nucleic acid sequence encoding VP16;
   (d) a nucleic acid sequence encoding ICP0;
   (e) a nucleic acid sequence encoding ICP4; and
   (f) a nucleic acid sequence encoding ICP27.
(2) The packaging cell line of aspect 1, wherein the nucleic acid sequence encoding rtTA is under the control of a constitutive promoter.
(3) The packaging cell line of aspect 2, wherein the constitutive promoter is CAG.
(4) The packaging cell line of any one of aspects 1-3, wherein the nucleic acid sequence encoding VP16 is under the control of a Tet-On system.
(5) The packaging cell line of any of aspects 1-4, wherein VP16 is expressed as a fusion polypeptide comprising a reporter.
(6) The packaging cell line of any one of aspects 1-5, wherein the nucleic acid sequence encoding ICP0 is under the control of a Tet-On system.
(7) The packaging cell line of any one of aspects 1-6, wherein the nucleic acid sequence encoding ICP0, nucleic acid sequence encoding VP16, the nucleic acid sequence encoding ICP4, helper virus genome, and/or the nucleic acid sequence encoding IPC27 is in a safe harbor site of the packaging cell line.
(8) The packaging cell line of any one of aspects 1-7, wherein the nucleic acid sequence encoding ICP4 is under the control of its cognate viral promoter.
(9) The packaging cell line of any one of aspects 1-8, wherein the nucleic acid sequence encoding ICP27 is under the control of its cognate viral promoter.
(10) The packaging cell line of any one of aspects 1-9, wherein the helper virus genome does not contain an origin of replication (ori) or a packaging signal (pac).
(11) The packaging cell line of aspect 10, wherein the helper virus genome does not contain oriL/S.
(12) The packaging cell line of any one of aspects 1-11, wherein the helper virus genome does not express one or more of ICP0, ICP4, and ICP27.
(13) The packaging cell line of aspect 12, wherein the helper virus genome comprises an inactivating deletion of one or more of ICP0, ICP4, and ICP27.
(14) The packaging cell line of aspect 13, wherein the helper virus genome comprises inactivating deletions of ICP0, ICP4, and ICP27.
(15) The packaging cell line of aspect 14, wherein at least one of the inactivating deletions is a complete deletion of the coding sequence of one or more of ICP0, ICP4, and ICP27.
(16) The packaging cell line of any one of aspects 12-15, wherein the helper virus genome does not contain nucleic acid sequences encoding ICP0, ICP4, and ICP27.
(17) The packaging cell line of any one of aspects 1-16, wherein the helper virus genome does not contain an internal repeat region (joint).
(18) The packaging cell line of any one of aspects 1-17, wherein the cell line is a mammalian cell line.
(19) A method of producing a herpes simplex virus (HSV) amplicon particle comprising transfecting the packaging cell line of any one of aspects 1-18 with an amplicon vector comprising an origin of replication (ori) and a packaging signal (pac).
(20) The method of aspect 19, further comprising isolating the HSV amplicon particle from the packaging cell line.
(21) The method of aspect 19 or 20, wherein the HSV amplicon particle is an HSV-1 amplicon particle.
(22) The method of any one of aspects 19-21, wherein the amplicon vector further comprises at least one transgene.
(23) An HSV amplicon particle produced by the method of any one of aspects 19-22.
(24) The HSV amplicon particle of aspect 23, comprising the amplicon vector surrounded by HSV capsid, tegument, and envelope proteins.
(25) A cell comprising the HSV amplicon particle of aspect 23 or 24.
(26) A kit comprising the HSV amplicon particle of aspect 23 or 24.
(27) A kit for preparing an HSV amplicon particle comprising (i) the packaging cell line of any one of aspects 1-18 and (ii) an amplicon vector comprising an origin of replication (ori) and a packaging signal (pac).
(28) The kit of aspect 27, wherein the amplicon vector further comprises at least one transgene.
(29) A method of expressing a transgene in a cell comprising infecting the cell with an HSV amplicon particle produced by the method of aspect 22, such that the at least one transgene is expressed within the cell.
(30) The method of aspect 29, wherein the cell is in vivo.
(31) The method of aspect 30, wherein the cell is a liver cell, lung cell, epithelial cell, neural cell, cardiac cell, muscle cell, stem cell, or cancer cell.
(32) The method of aspect 31, wherein the cell is in vitro.
(33) The method of any of any one of aspects 29-32, wherein the cell is a human cell.
(34) A method of treating a disease or condition in a subject, comprising administering an HSV amplicon particle produced by the method of aspect 22 to the subject, in an amount and at a location sufficient to infect cells of the subject such that the at least one transgene is expressed in the subject.
(35) The method of aspect 34, wherein the disease or condition is a cancer and the at least one transgene encodes an agent that enhances tumor killing activity.
(36) The method of aspect 34, wherein the disease or condition is muscular dystrophy, cardiovascular disease, neurodegenerative disease, chronic pain, lung disease, or hemophilia.
(37) The method of any one of aspects 34-36, wherein the subject is a human.
(38) A composition comprising the HSV amplicon particle of aspect 23 or 24 and a pharmaceutically acceptable carrier.

The following examples further illustrate aspects of the invention but, of course, should not be construed as in any way limiting its scope.

### Example 1

This example demonstrates the production of a complementing cell line for amplicon particle generation.

The manufacturing limitations of HSV amplicons can be solved by engineering a specialized amplicon packaging cell line. Instead of using a helper virus for amplicon production, the HSV genes required to produce amplicon particles are permanently locked into one or several chromosomes of the packaging cell line where the viral genes are maintained in a silent state until induced by drug at the time of amplicon DNA transfection (FIG. 1, left). Using site specific recombination, the bulk of the helper virus genes are inserted in the packaging cell line at one of several “safe harbor” loci identified in the mammalian genome (see Mitchell et al., J. Vis. Exp., (99): e52941 (2015); and Papapetrou et al., Mol. Ther., 24(4): 678-684 (2016)).

These genes are silent until activated by a drug-controlled master switch (Tet-On VP16) that acts from a different safe harbor site to initiate the transcription of two separately integrated viral genes (ICP4, ICP27) that in turn set in motion the complete natural cascade of gene expression from the integrated bulk of helper virus genes. This process is enhanced by drug-mediated induction of another separately integrated viral gene, ICP0, under control of the Tet-On promoter.

Together, this cell system provides the array of viral functions required for efficient amplicon DNA amplification and packaging into amplicon particles.

The packaging cell line can contain at different chromosomal sites (1) a helper virus genome without ori/pac sequences and without the ICP0, ICP4, and ICP27 genes; (2) rtTA controlled by a constitutive promoter like CAG; (3) Tet-On VP16 (as a VP16-2A-GFP fusion to monitor induction); (4) Tet-On ICP0; and (5) ICP4 and ICP27 under their respective cognate viral promoters.

Upon transfection of the amplicon DNA into this packaging cell line (FIG. 1, right), the master switch is turned on by drug addition and infectious amplicon particles comprising, consisting essentially of, or consisting of one or more tandem copies of the amplicon DNA surrounded by the HSV capsid, tegument, and envelope proteins are produced (FIG. 1, lower right).

The complete virus shell is needed for efficient amplicon particle cell attachment/entry, intracellular transport, uncoating, and DNA delivery to the nucleus. Only the “free-standing” transfected amplicon DNA contains the necessary signals for DNA replication (“ori”) and packaging (“pac”) while all HSV helper virus sequences are locked into the cellular genome so that the system ensures the production of amplicon particles entirely free of contaminating viral gene sequences. Manufacturing of commercially relevant viral titers in the range of 10⁸ to 10¹⁰ Transduction Units (TU) not previously achievable with helper-dependent HSV systems are possible by repeated passaging.

Thus, using the initial amplicon yield, the amplicon production system allows particle amplification by infection of increasingly large cell numbers similar to production of regular HSV stocks as the particles produce plaques on the packaging cell line or support running cycles of infection in liquid culture.

### Example 2

This example demonstrates amplicon vector generation using the inventive packaging cell line.

Amplicon DNA used for transfection of packaging cell lines is an E. coli plasmid carrying the coding sequences for one or more products of interest, such as proteins or non-coding RNA, along with applicable regulatory sequences (“payload”). In addition, the plasmid insert contains one copy each of an HSV ori and pac sequence on either side of the payload DNA (FIG. 1, right).

Upon transfection into the packaging cell line and activation of the integrated HSV helper genes, these plasmids replicate like episomal HSV genomes, forming head-to-tail concatemers of the entire plasmid DNA (Fraefel et al., Methods Mol. Biol., 2060: 91-109 (2020)). These concatemers are cut by the packaging machinery to generate linear fragments of the right size (˜150±10 kb) for uptake into capsids.

However, in such a method, bacterial sequences corresponding to the plasmid backbone are included in the packaged DNA, reducing the number of tandem copies of the payload present in each ˜150-kb concatemer. These bacterial sequences are recognized as foreign in mammalian host cells, eliciting an innate response that can substantially diminish short- and especially long-term payload expression (Suzuki et al., J. Virol., 80(7): 3293-3300 (2006)).

To circumvent this drawback, amplicon DNAs can be produced as minicircles (FIG. 1, right) generated from recombinant plasmids by inducible bacteriophage (e.g., ΦC31) integrase-mediated recombination in specialized bacteria between compatible integrase recognition (“att”) sites at both ends of the plasmid insert (payload+ori/pac); the reciprocal product (circular plasmid backbone) is subsequently degraded by inducible expression of a restriction enzyme that lacks recognition sites in the insert but cuts at repeated sites in the backbone (System Biosciences, systembio.com/wp-content/uploads/Minicircle-production-manual.pdf).

In principle, payloads that are too large for efficient plasmid-based replication and minicircle production can be produced as bacterial artificial chromosomes (BACs), followed by elimination of the bacterial sequences by inducible integrase-mediated “looping-out” and selective degradation of the bacterial elements flanked by compatible att sites, similar to plasmid-derived minicircle production but requiring a BAC-compatible minicircle E. coli strain.

The large payload HSV-mediated gene delivery afforded by the invention enables the use of full-length human gene loci, including introns and transcription regulatory sequences, such as native enhancers and ectopic chromatin boundary/insulator/anti-silencing elements, allowing for natural spatial and temporal control of therapeutic gene expression. The design of amplicon-based defective-gene complementation can take advantage of natural gene regulation for recessive genetic diseases and combination of gene editing with gene complementation to treat dominant genetic diseases.

Large payloads also can be used as gene editing tools for defective gene correction using amplicon DNA as the repair template. The amplicon particles can be further modified to target infection to non-cognate cellular receptors for payload delivery to specific cell types. The use of cell-specific promoters and target sites for resident microRNAs can further limit transgene expression.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A packaging cell line comprising:
(a) a helper virus genome;
(b) a nucleic acid sequence encoding reverse tetracycline transactivator (rtTA);
(c) a nucleic acid sequence encoding VP16;
(d) a nucleic acid sequence encoding ICP0;
(e) a nucleic acid sequence encoding ICP4; and
(f) a nucleic acid sequence encoding ICP27.

2. The packaging cell line of claim 1, wherein the nucleic acid sequence encoding rtTA is under the control of a constitutive promoter.

3. (canceled)

4. The packaging cell line of claim 1, wherein the nucleic acid sequence encoding VP16 is under the control of a Tet-On system.

5. The packaging cell line of claim 1, wherein VP16 is expressed as a fusion polypeptide comprising a reporter.

6. The packaging cell line of claim 1, wherein the nucleic acid sequence encoding ICP0 is under the control of a Tet-On system.

7. The packaging cell line of claim 1, wherein the nucleic acid sequence encoding VP16, the nucleic acid sequence encoding ICP0, the nucleic acid sequence encoding ICP4, helper virus genome, and/or the nucleic acid sequence encoding IPC27 is in a safe harbor site of the packaging cell line.

8. The packaging cell line of claim 1, wherein the nucleic acid sequence encoding ICP4 is under the control of its cognate viral promoter.

9. The packaging cell line of claim 1, wherein the nucleic acid sequence encoding ICP27 is under the control of its cognate viral promoter.

10. The packaging cell line of claim 1, wherein the helper virus genome does not contain an origin of replication (ori) or a packaging signal (pac).

11. (canceled)

12. The packaging cell line of claim 1, wherein the helper virus genome does not express one or more of ICP0, ICP4, and ICP27.

13-16. (canceled)

17. The packaging cell line of claim 1, wherein the helper virus genome does not contain an internal repeat region (joint).

18. The packaging cell line of claim 1, wherein the cell line is a mammalian cell line.

19. A method of producing a herpes simplex virus (HSV) amplicon particle comprising transfecting the packaging cell line of claim 1 with an amplicon vector comprising an origin of replication (ori) and a packaging signal (pac).

20-22. (canceled)

23. An HSV amplicon particle produced by the method of claim 19.

24. (canceled)

25. A cell comprising the HSV amplicon particle of claim 23.

26. A kit comprising the HSV amplicon particle of claim 23.

27. A kit for preparing an HSV amplicon particle comprising (i) the packaging cell line of claim 1 and (ii) an amplicon vector comprising an origin of replication (ori) and a packaging signal (pac).

28. (canceled)

29. A method of expressing a transgene in a cell comprising infecting the cell with the HSV amplicon particle of claim 23, wherein the wherein the amplicon vector further comprises at least one transgene, such that the at least one transgene is expressed within the cell.

30-33. (canceled)

34. A method of treating a disease or condition in a subject, comprising administering the HSV amplicon particle of claim 23 to the subject, wherein the wherein the amplicon vector further comprises at least one transgene, in an amount and at a location sufficient to infect cells of the subject such that the at least one transgene is expressed in the subject.

35-37. (canceled)

38. A composition comprising the HSV amplicon particle of claim 23 and a pharmaceutically acceptable carrier.
